(19) United States
(12) Patent Application Publication
(10) Pub. No.: US 2007/0080062 A1
(43) Pub. Date: Apr. 12, 2007

(54) Coated metal structures and methods of making and using thereof

(76) Inventors: Cindy K. Harnett, Livermore, CA (US); Tyrone F. Hill, Cambridge, MA (US); Michael P. Kanouff, Livermore, CA (US)

Correspondence Address: SMITH, GAMBRELL & RUSSELL (SNL), 1850 M STREET, NW, # 800, WASHINGTON, DC, 20036, US

(21) Appl. No.: 11/240,396

(22) Filed: Oct. 3, 2005

(51) Int. Cl. C07K 1/26; B32B 15/04

(52) U.S. Cl. 204/450; 428/457; 428/469

(57) ABSTRACT

Disclosed herein are methods and devices for applying two or more mobilization fields in a microchannel using a multifunctional structure and manipulating particles and fluids based on more than one characteristic. Specifically, a fluidic channel comprising at least one metal structure having a coat comprising a conductive material, an insulative material, a semi-conductive material, or a combination thereof, is disclosed. Also disclosed are methods for manipulating or assaying a particle in a sample which comprises subjecting the sample to a fluidic channel comprising at least one metal structure having a coat comprising a conductive material, an insulative material, a semi-conductive material, or a combination thereof and inducing at least one mobilization field such as a magnetic field, an electroosmotic field, an insulative dielectrophoresis (iDEP) field, which iDEP field may be an iDEP trapping field or an iDEP streaming field, or a combination thereof.

### ACKNOWLEDGEMENT OF GOVERNMENT SUPPORT

The present invention was made by employees of Sandia National Laboratories. The Government has certain rights in the invention.

### BACKGROUND OF THE INVENTION

1. Field of the Invention.

The present invention relates to microfluidics, micro-total-analysis systems (μTAS) and micro-electro-mechanical systems (MEMS). In particular, the present invention relates to microfluidic pumps and mixers.

2. Description of the Related Art.

The ability to transport fluids in micron-sized channels is essential for many emerging technologies, such as in vivo drug delivery devices, micro-electro-mechanical systems (MEMS), and micro-total-analysis systems (μTAS). New methods for the rapid mixing of inhomogeneous fluids in micron-scale devices are also required, since the absence of turbulent mixing on these small length scales implies that mixing occurs by molecular diffusion alone. This typically takes from seconds to minutes which is far too slow for envisioned applications. New technologies are thus required for the manipulation, transport and mixing of fluids on these small length scales.

Although MEMS-based mechanical pumps with moving parts have recently been developed, including peristaltic pumps, a variety of non-mechanical pumping strategies without moving parts have been used, e.g. based on electrical fields, thermal gradient, electrochemical reactions, surface tensions gradients, and patterned surfaces. Non-mechanical strategies for fluid manipulation become more efficient at very small scales because they are driven by surface phenomena. Moreover, they can be much cheaper to implement than mechanical MEMS-based strategies because they take advantage of nano-scale chemical effects already exhibited by many fluids used in biomedical and chemical engineering applications. They can also possess fewer parts, and are better suited for flexible devices, such as microfluidic fibers.

Perhaps the most popular non-mechanical fluid manipulation strategy is based on the phenomena of electro-osmosis, i.e. the fluid slip at a solid-electrolyte interface induced by a tangential electric field. The fluid is set into motion by strong electrostatic body forces exerted by excess ionic charge in diffuse boundary layers of thickness λ=1−100 nm near a solid interface. This effect, which has been studied extensively for more than a century in colloidal science and electrochemistry, is well suited for biomedical applications because the majority of bodily fluids, such as blood or lymph, are electrolytes with comparable ionic strengths. Moreover, the working electrode imposing spatially or temporally varying electric fields can be easily and cheaply built into microchannels with existing silicon-based micro-fabrication technology. Driving fluids with electric fields also facilitates integration with logic circuits for sensing and integration microfluidic devices.

U.S. Patent Publication No. 20030164296 described induced-charge electroosmosis (ICEO) using a device comprising at least one conductor element, i.e. a solid metal post, that is placed in at least one specific location in the device to provide a defined electrical field, thereby resulting in electroosmotic flows such that an electrolyte fluid is driven across a microchannel. Unfortunately, fabrication of microfluidic devices having solid metal posts is cumbersome and expensive. Additionally, the device and method described are limited to electrolyte fluids and electroosmotic flows. Therefore, the methods and devices described in the publication are unsuitable for assaying complex fluids and complex analytes.

Further the microfluidic pumps and mixers in the prior art may be only used as one or the other, i.e. a pump can only function as a pump. No where does the prior art provide a microfluidic device having a structure which may act as a pump, a mixer, and a sorter (or separator). Additionally, the microfluidic separators and sorters in the prior art only manipulate or separate particles and fluids based on a single characteristic.

Thus, a need exists for ICEO devices that are relatively easy and economical to fabricate as well as methods and devices that allow analysis of complex fluids and complex analytes. A need also exists for microfluidic devices which have structures that are multifunctional and may be used to manipulate particles and fluids based on more than one characteristic.

### SUMMARY OF THE INVENTION

The present invention provides methods and devices for applying two or more mobilization fields in a microchannel using a multifunctional structure and manipulating particles and fluids based on more than one characteristic.

In some embodiments, the present invention provides a fluidic channel comprising at least one metal structure having a coat comprising a conductive material, an insulative material, a semi-conductive material, or a combination thereof. In some embodiments, the metal structure is a structure comprising an insulative material, such as glass or a polymer and a metal coat. In some embodiments, the metal structure comprises solid metal. In some embodiments, the metal structure comprises a magnetic material, a paramagnetic material, or a ferromagnetic material. In some embodiments, the coat is about 1 nm to about 5000 nm thick. In some embodiments, the metal structure provides a magnetic field or an electroosmotic field under an applied electrical current. In some embodiments, the coat provides a mobilization field under an applied electrical current, which mobilization field is an electroosmotic field, an insulative dielectrophoresis (iDEP) field, or a combination thereof. The iDEP field may be an iDEP trapping field or an iDEP streaming field.

In some embodiments, the present invention provides a method of manipulating or assaying a particle in a sample which comprises subjecting the sample to a fluidic channel comprising at least one metal structure having a coat comprising a conductive material, an insulative material, a semi-conductive material, or a combination thereof and inducing at least one mobilization field. In some embodiments, the metal structure is a structure comprising an insulative material, such as glass or a polymer and a metal coat. In some embodiments, the metal structure comprises solid metal. In some embodiments, the metal structure comprises a magnetic material, a paramagnetic material, or a ferromagnetic material. In some embodiments, the coat is about 1 nm to about 5000 nm thick. In some embodiments, the mobilization field is an electroosmotic field. In some embodiments, the metal structure comprises a magnetic material and the mobilization field is a magnetic field. In some embodiments, wherein the coat comprises a conductive material, the method further comprises inducing an electroosmotic field. In some embodiments, wherein the coat comprises an insulative material, the method further comprises inducing an iDEP field, which iDEP field may be an iDEP trapping field or an iDEP streaming field. In some embodiments, wherein the coat comprises a semi-conductive material, the method further comprises inducing an electroosmotic field before or after inducing an iDEP field, which iDEP field may be an iDEP trapping field or an iDEP streaming field. In some embodiments, the mobilization fields are induced concurrently or overlap.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIG. 1 illustrates ICEO phenomenon, wherein charges separate near a polarized metal object and are moved by the field, dragging the surrounding fluid (electro-osmosis).

FIG. 2 schematically shows a method for making metal structures (metal coated polymeric structures). In Step 1, a patterned substrate is cleaned with acetone/IPA and dehydrated at about 80° C. for about 30 minutes. Then a layer of Ti and a layer of Au are applied. In Step 2, resist is applied. In Step 3, the coated substrate is exposed and developed. In Step 4, the Au layer is etched and then the Ti layer is etched. In Step 5, the resist is removed.

FIG. 3 is a top view photograph of an example of the metal structures in a microchannel. The diameters of the metal structures in the middle of the array are about 200 μm. The metal structures at the ends of the array are an example of petal shaped metal structures.

FIG. 4 is a representation of a cross-sectional view of the metal structures of FIG. 3.

FIG. 5 shows streamlines that were calculated from video microscopy data superimposed on an image of the metal coated polymeric structures.

FIG. 6A is a grayscale image created from a single snapshot of a mixture of different sized red and green microparticles inside a channel, wherein large (500 nm diameter) red particles are highlighted

FIG. 6B, is a grayscale image created from a single snapshot of a mixture of different sized red and green microparticles inside a channel, wherein the small (200 nm diameter) green particles are highlighted.

FIG. 7 shows mixing vortices forming over alternating half metal coated polymeric structures when an AC field is applied.

FIG. 8A shows an uncoated metal structure and its cross-section according to the present invention.

FIG. 8B shows a coated structure and its cross-section. The structure may be an insulating structure, e.g. made of an insulative material, with a metal coat to give an embodiment of a metal structure according to the present invention. Alternatively, the structure may be a metal structure with a coat that may be a conductive material, an insulative material, a semiconductive material, or a combination thereof.

FIG. 8C shows an example of a partially coated structure and its cross-section. The structure may be an insulating structure, e.g.. made of an insulative material, a metal coat to give an embodiment of a metal structure according to the present invention. Alternatively, the structure may be a metal structure with a coat that may be a conductive material, an insulative material, a semiconductive material, or a combination thereof.

FIG. 8D shows a metal structure having a coat of an insulative material and a coat of a semiconductive material. The metal structure may be a solid metal structure or an insulating structure, e.g. made of an insulative material, having a metal coat.

FIG. 9 is an oversimplification of an example of a device according to the present invention.

### DETAILED DESCRIPTION OF THE INVENTION

The present invention provides devices comprising metal coated structures and methods of making and using thereof. In particular, the present invention provides microfluidic devices comprising microchannels having metal coated structures. As used herein, “microfluidic” refers to a system or device having one or more fluidic channels, conduits or chambers that are generally fabricated at the millimeter to nanometer scale. As used herein, “channel” refers to a structure wherein a fluid may flow. A channel may be a capillary, a conduit, a strip of hydrophilic pattern on an otherwise hydrophobic surface wherein aqueous fluids are confined, and the like. Thus, the “microfluidic channels” or alternatively referred to herein as “microchannels” of the present invention generally have cross-sectional dimensions ranging from about 10 nm to about 1 mm.

The present invention also provides methods and devices for creating different types of mobilization fields including electric fields, magnetic fields, electromagnetic fields, or combinations thereof. As used herein, the term “mobilization field” refers to any force field that influences a particle from one point to another point. Mobilization fields include hydrodynamic flow fields produced by pressure differences, gravity, linear or centripetal acceleration; electrokinetic flow fields; magnetophoretic flow fields; thermophoretic flow fields; electric field; magnetic fields; electromagnetic fields; and others known in the art. Such fields may be used to manipulate fluids and analytes in the fluids by dielectrophoresis (DEP) and insulator based dielectrophoresis (iDEP), induced-charge electroosmosis (ICEO), magnetism, or a combination thereof. The manipulations include mixing, concentrating, separating, isolating, transporting, filtering, and rotationally orienting particles in a fluid.

As used herein, “concentrating” refers to the reduction of fluid volume per particle in the fluid. When the methods and devices are used to concentrate a fluid, particles in one portion of the fluid become “concentrated” and that particles in the second portion of the fluid become “diluted”.

Fluids and analytes may be assayed and characterized by the manner and extent the fluid or analyte is manipulated by a given field. Thus, the present invention provides methods and devices for assaying fluids and analytes in a sample. As used herein, “assaying” is used interchangeably with “detecting”, “measuring”, “monitoring” and “analyzing”.

As used herein, a “fluid” refers to a continuous substance that tends to flow and to conform to the outline of a container such as a liquid or a gas. Fluids include saliva, mucus, blood, plasma, urine, bile, breast milk, semen, water, liquid beverages, cooking oils, cleaning solvents, ionic fluids, air, and the like. Fluids can also exist in a thermodynamic state near the critical point, as in supercritical fluids. If one desires to test a solid sample for a given analyte according to the present invention, the solid sample may be made into a fluid sample using methods known in the art. For example, a solid sample may be dissolved in an aqueous solution, ground up or liquefied, dispersed in a liquid medium, melted, digested, and the like. Alternatively, the surface of the solid sample may be tested by washing the surface with a solution such as water or a buffer and then testing the solution for the presence of the given analyte.

As used herein, an “analyte” refers to a particle that may be natural or synthetic and includes compounds and biomolecules, such as amino acids, peptides, proteins, nucleotides, nucleic acids, carbohydrates, lipids, cells, viral particles, bacteria, spores, protozoa, yeast, mold, fungi, pollen, diatoms, and the like, and ligands, supermolecular assemblies, catalytic particles, zeolites, and the like.

As provided herein, fluids and particles, including analytes, are manipulated by exploiting their physical, electrical and chemical properties. The devices of the present invention allow more than one mobilization field to be employed to manipulate fluids and particles. Since more than one mobilization field may used, the present invention provides methods and devices for manipulating complex fluids and complex analytes. As used herein, a “complex fluid” or a “complex analyte” refers to a fluid or analyte which have two or more properties that are manipulated differentially by more than one mobilization field. For example, a complex fluid may contain a first analyte having a surface charge which allows it to be manipulated in a given electric field or a given electromagnetic field and a second analyte having a magnetic force which allows it to be manipulated in a given magnetic field or a given electromagnetic field.

It should be noted that the insulating structures and metal structures provided herein are not limited to a given shape such as a columnar like protrusion but encompass various shapes including cubes, pyramids, spheres, and the like, as well as ridges and valleys such as those described in U.S. Patent Publication No. 20050072676, which is herein incorporated by reference.

A. Metal Coated Insulating Structures

Nonlinear electrokinetic phenomena provide an extensive repertoire of fluid flow effects, including microvortex generation and rectification of flow driven by alternating current (AC) electric fields. See Ajdari, A. (2000) Phys. Rev. E 61, R45; and Ramos et al. (1999) J. Colloid Interface Sci. 217, 420, which are herein incorporated by reference. The phenomenon of induced-charge electroosmosis (ICEO) has been applied to the problem of microfluidic mixing and pumping. See Bazant & Squires (2004) Phys. Rev. Lett. 92:066101/1-4; and Bazant & Squires (2004) J. Fluid Mech. 509:217, which are herein incorporated by reference. Unfortunately, solid metal posts as described by Bazant & Squires are cumbersome and expensive to manufacture in microfluidic devices.

Therefore, the present invention provides microfluidic devices having insulating structures coated with metal which produce a nonuniform zeta potential under an applied electric field and are suitable for ICEO methods and applications. As used herein, the word “conductivity” is used to describe the ease of flow of both conduction and displacement current. It is often mathematically described as a complex number that varies with the frequency of the applied electric field. Similarly, “conduction” is used to describe both conventional conduction and conduction of displacement currents. As used herein, “insulative” and “conductive” refers to the relative conductivity of the described item with respect to the fluid or analyte being manipulated according to the methods of the present invention. Thus, insulating structures are those made of a material that are less conductive than the surrounding fluid or analyte.

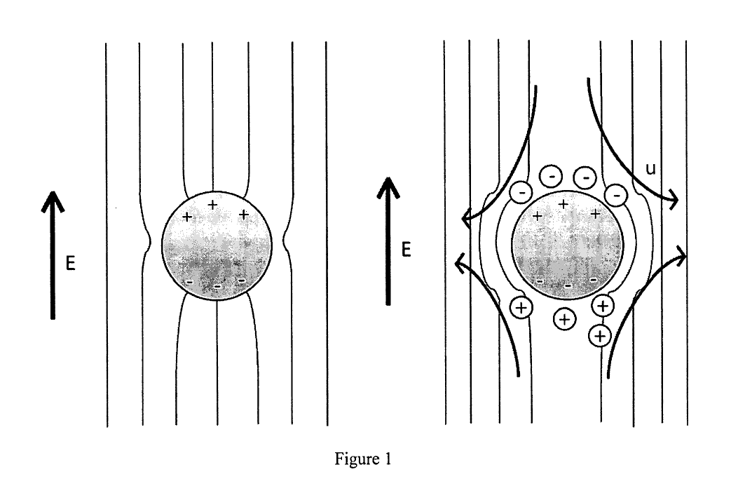

FIG. 1 exemplifies charges within a fluid that rearrange in response to the surface charge at the metal coated insulating structures according to the present invention. Local electrokinetic pumping at these surfaces then produces tangential flows at the edges of the structures. When the structures are configured in an array, these flows combine to form microvortices surrounding each structure. Because both the sign of the surface charge and the direction of the field are reversed when the field sign changes, the local pumping direction stays constant under an AC field. As described herein, the microvortices produce electronically-controllable mixing flow patterns, and are observed to concentrate particles from the surrounding fluid into different regions based on their size. Thus, the present invention not only provides methods and devices for traditional ICEO applications, but also methods and devices for producing microvortices and concentrating particles according to size around the regions of the metal coated insulating structures.

A1. Fabrication

The metal coated insulating structures according to the present invention may be made by various methods known in the art. Preferred methods of making include the following:

a. Electron-Beam Lithography (EBL) Process Flow Method

First, a substrate, such as a glass wafer, is obtained. Microfluidic channels and nanostructures, such as posts, are formed on the substrate using methods known in the art. The substrate is then coated with a uniform-thickness over the surface topography, preferably regardless of orientation of the surface normal with metal. Then a desired pattern in the metal is formed using methods known in the art such as chemical etching through a polymer layer that has been patterned by electron beam lithography.

Then the substrate is cleaned for about 10 minutes using methods known in the art such as piranha cleaning (e.g. 3:1 sulfuric acid/hydrogen peroxide at about 100° C.).

Next, the substrate is subjected to a dehydrating bake at about 120° C. for about 10 minutes. Then a sputter coat of about 1000 Å to about 2000 Å of Cr is deposited.

The substrate is baked on a hotplate at about 90° C. for about 5 minutes and then about 30 nm to about 50 nm of 495K PMMA (2% solution in dichlorobenzene, MicroChem Inc, Newton, Mass.) is deposited on the substrate using methods known in the art such as spin coating. The substrate is then hard baked at about 125° C. for about 5 minutes.

A pattern, aligned with the metal coated insulating posts under the PMMA layer, is then formed in the PMMA layer using methods known in the art such as electron beam lithography, e.g. about 100 to about 200 μCoulombs/cm². Then PMMA is removed from the patterned areas by, for example, developing for about 1 minute in 1:3 MIBK/IPA (methylisobutylketone/isopropanol) solution and rinsing with IPA.

The Cr is etched for 2 minutes in chromium mask etchant (Transene Company Inc., Danvers, Mass.) to selectively remove the unwanted metal from the electron-beam exposed areas. In areas that are still covered by unexposed PMMA, the metal is protected from the etchant and will remain intact. PMMA is then removed with acetone, thereby resulting in a patterned metal film on top of the insulating structures.

b. Photolithography Method

This method is preferably used to create metal patterns on a substrate sensitive to high processing temperatures, e.g. above 100° C., such as a polymer substrate, however, other suitable substrates such as glass may be used.

Generally, a substrate, such as a Zeonor® wafer, is obtained. Microfluidic channels and nanostructures are formed in the substrate using methods known in the art. The substrate is cleaned with acetone/IPA, then dehydrated at about 80° C. for about 30 minutes. Then a coat of about 750 Å of Ti and a coat of about 1500 Å of Au are deposited on the substrate using methods known in the art such as sputter coating.

Then a coat of about 10 μm of positive photoresist, such as Microposit SJR 5740 resist (Rohm and Haas Electronic Materials LLC, Marlborough, Mass.) is deposited at about 2200 rpm. The substrate is then baked at about 80° C. for about 10 minutes.

A photomask is aligned to the topographic features on the substrate, and then the photoresist is exposed and developed using methods known in the art, e.g UV light and Microposit developer (Rohm and Haas Electronic Materials LLC, Marlborough, Mass.). The photomask may be a pattern of chrome on glass, or other opaque pattern on a clear substrate, and the like. Suitable photomasks include those commercially available (Photo-Sciences, Inc., Torrance, Calif.) or made using methods known in the art, such aschrome-etching. The Au layer is etched using a KI/I₂/H₂O solution for about 30 seconds and the Ti layer is etched with an HF/HCI/H₂O solution for about 30 seconds. The resist is then removed by spinning on acetone.

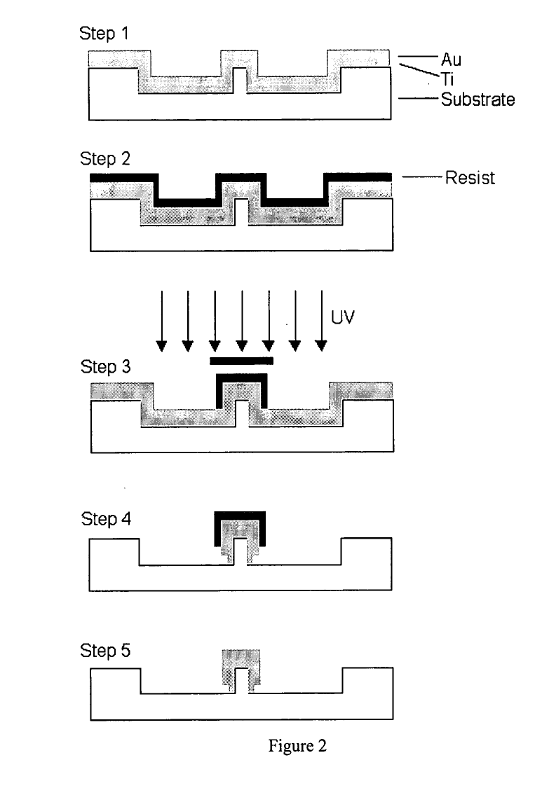

FIG. 2 shows a schematic of an example of how the metal coated insulating structures may be fabricated. As shown in FIG. 2, Zeonor® 1020 polymer substrates (Zeon Chemicals, Louisville, Ky.) were fabricated by injection molding on a nickel template which was created by electroplating a wet-etched glass wafer using methods known in the art to produce a polymer wafer with channels and arrays of insulating structures. The insulating structures had beveled edges typical of wet-etched glass.

Isolated conductive areas were then patterned on top of individual structures. The entire wafer was coated with about a 20 nm thick titanium adhesion layer and about a 200 nm gold conductive layer using methods known in the art. Photoresist SJR 5740, (Rohm and Haas Electronic Materials LLC, Marlborough, Mass.) was patterned to protect only the areas on the insulating structures. The exposed metal was removed by wet chemical etching using methods known in the art. Exposure to high temperatures, e.g. greater than 100° C., and harsh solvents should be avoided when Zeonor® is used.

After metal patterning, fluid access holes were drilled in the Zeonor® substrate at each end of the channel. Channels were capped with poly(dimethylsiloxane) (PDMS) lids which were cast at about 2 mm thick using methods known in the art. To aid lid adhesion, both the metal coated Zeonor® substrate and PDMS lid were exposed to an oxygen plasma at 100 W for one minute immediately before bonding.

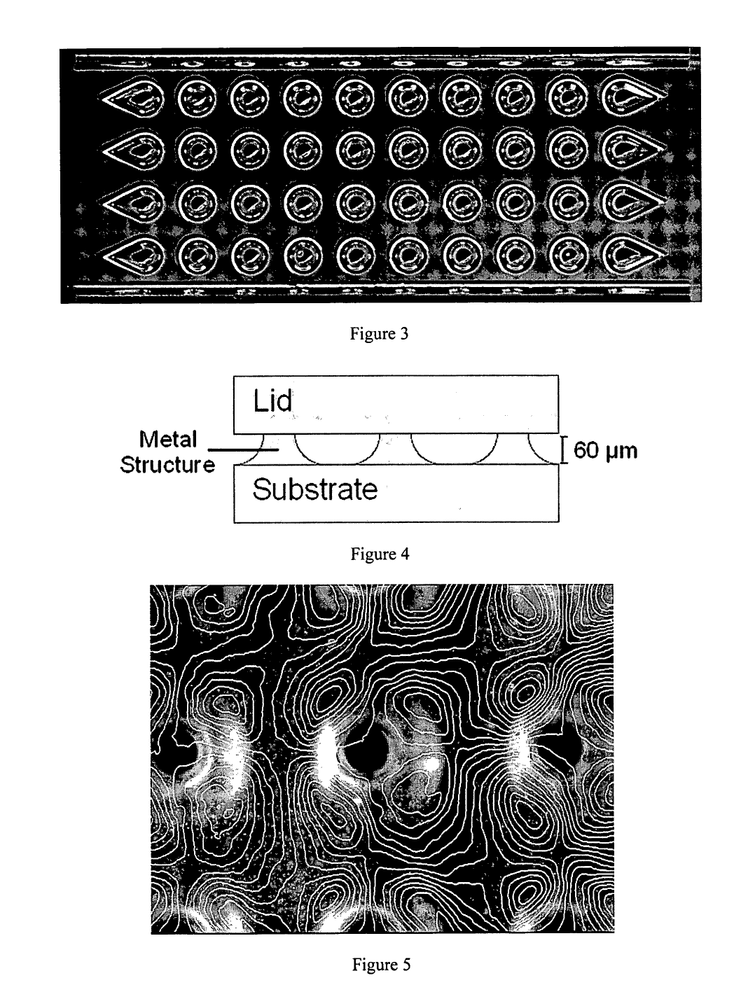

FIG. 3 is a close-up image of a channel having metal coated Zeonor® structures in which the sides and tops of 60 μm tall×200 μm wide circular structures have been covered with a gold film. FIG. 4 shows a representation of the cross-section of FIG. 3. Various channel designs having metal coated insulating structures were created on 4-inch diameter Zeonor® wafers, and elastomer lids were sealed to the channel sides and tops of the structures for mixing and pumping experiments.

A2. Experiments

Devices comprising the metal coated insulating structures were powered by an electric circuit which generated a square wave of variable frequency of about 20 to about 2500 Hz, peak-to-peak amplitude of up to about 300 V, and about a 50% duty cycle to eliminate DC components which would cause electrolysis and global electrokinetic pumping. Platinum wire electrodes were attached in open reservoirs at the ends of the 1 cm-long channels.

Solutions containing fluorescent, carboxyl-functionalized polystyrene tracer beads (Molecular Probes FluoSpheres®, Invitrogen Corporation, Carlsbad, Calif.) at 0.02% solids in 0.1 mM KCI were loaded into the channels for particle image velocimetry. The addition of 1 μl Triton-X 165 surfactant per 1 ml bead solution reduced bead attachment to the metal coated insulating structures without interfering with the electrokinetic flows.

FIG. 5 shows a set of streamlines that were calculated from video microscopy data. These streamlines have been superimposed on an image of the metal coated insulating structures. Closed streamlines indicate that near the metal coated insulating structures, particles become confined within individual vortices. Electrical conditions for this image were a square wave frequency of about 37 Hz, and peak-to-peak field strength of about 70 V/cm. The tracer particles were 200 nm diameter FluoSpheres®.

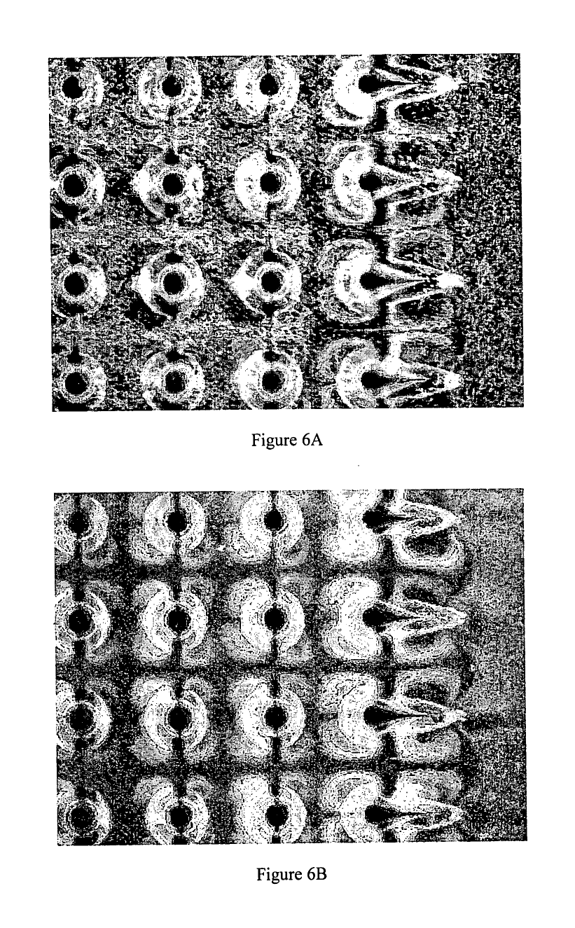

FIG. 6A and FIG. 6B are a pair of grayscale images created from a single snapshot of a mixture of different sized red and green microparticles (FluoSpheres®) inside the channel, about three seconds after starting the AC signal. In FIG. 6A, the large (500 nm diameter) red particles are highlighted and show in the gray scale image as light gray to white areas, and in FIG. 6B, the small (200 nm diameter) green particles are highlighted show in the gray scale image as light gray to white areas. The small particles are observed to concentrate closer to the centers of the microvortices than the large particles. The applied electrical signal was about a 28 Hz square wave with a field strength of about 155 V/cm peak to peak. Vortex speed and size were observed to reduce with increased frequency, from maximum strength near DC, to imperceptible at about 2000 Hz.

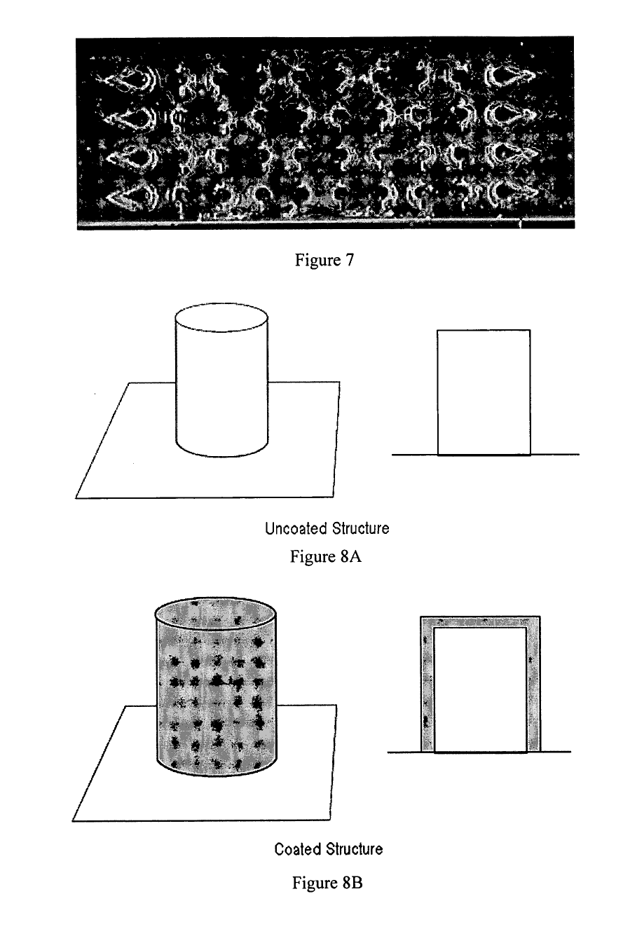

FIG. 7 shows mixing vortices forming over alternating half metal coated insulating structures when an AC field was applied. As shown in FIG. 7, the channel width was about 1 mm, the applied field was about 500 V, about 50 Hz across electrodes placed in reservoirs at each end of the channel and the fluid was 0.1 mM KCI with about 0.025% green fluorescent 200 mn diameter polystyrene tracer spheres (FluoSpheres®) by weight.

A3. Analysis and Discussion

The quadrupolar vortices observed in FIG. 5 produce local stirring. To use the effect for mixing across the entire channel, particles need to be moved out of their original vortex. One skilled in the art may readily effect mixing across an entire channel by designing device architectures and metal coated insulating structure arrays to apply a desired pressure-driven flow, a desired electroosmotic flow, and the like. For example, the shape, such as a petal shape, of the insulating structures may readily be selected to produce desired mixing vortices and flows. Additionally, the insulating structures may readily be partially coated in a pattern or on a particular side with metal in order to effect a desired result. AC operation of asymmetric shaped metal coated insulating structures was not observed to produce strong pumping. However, small pumping effects may be amplified by increasing the pumping surface area, similar to high-pressure DC electrokinetic pumps made from packed submicron-diameter silica particles, using methods known in the art.

Therefore, the present invention provides microfluidic channels comprising metal coated insulating structures which rapidly polarize when an electric field is applied to the ends of the channel and methods of making thereof. The resulting non-uniform surface charge causes local electrokinetic pumping under the applied field, producing microvortices of about 100 μm diameter under alternating-current (AC) electric fields of about 30 to about 100 Hz and field strengths of about 300 V/cm. Further, the devices and methods of the present invention provided spatial separation between 200 nm and 500 nm polystyrene fluorescent particles. Therefore, the devices and methods of the present invention may also be used to separate or isolate particles of different sizes.

Azimuthal Electric Field

In some embodiments, an electromagnetic coil is fabricated within the metal structure to generate an azimuthal electric field and cause electroosmotic flow around the metal structure in a circular pattern for highly controlled mixing. In these embodiments, a coating is applied to the metal structure to create the desired surface charge for electroosmotic pumping. In preferred embodiments, the coating is made of a material having an immobilized surface charge such as silica or an organic coating with switchable surface charge such as propyl-N,N,N-trimethylammonium. See Tien, et al. (2001) Chem. Mater. 13:1124, which is herein incorporated by reference.

B. Conductor, Insulator, or Semiconductor Coated Metal Structures

The present invention also provides microfluidic devices comprising conductor, insulator, or semiconductor coated metal structures and methods of making and using thereof. In preferred embodiments, the metal structure is preferably solid. It is noted, however, that the metal structure may be a first metal coating over an insulating structure upon which a second coat is applied as provided in part A above. Metal structures coated with an insulator may be used for iDEP applications, metal structures coated with a conductor may be used for ICEO applications, metal structures coated with both in a given pattern may be used for ICEO mixing applications, and metal structures having a semiconductor coating may be used for combined iDEP and ICEO applications.

In preferred embodiments, the metal structure is made of a material that has properties that are different from the properties of the metal coating material to result in two or more mobilization fields. In some preferred embodiments, the metal structure is made of a magnetic material that will magnetize when a magnetic field is present, such as nickel. In preferred embodiments, the metal coating is made of a material that is chemically nonreactive or substantially resistant to oxidation and strong chemicals, such as gold or platinum. Suitable magnetic materials for the metal structures are paramagnetic materials such as magnesium, molybdenum, and tantalum, and the like; and ferromagnetic materials such as nickel, iron, cobalt, and the like; and composites and mixtures thereof. Preferred magnetic materials for the metal structures are paramagnetic since they will not remain magnetized when the external magnetic field is removed.

Suitable conductive materials for the conductor coating include metals such as gold, platinum, tantalum, and the like, organic conductors such as polypyrrole, and composites and mixtures thereof. Suitable insulative materials for the insulator coating or insulating structure include oxides, nitrides, polymers, plastics, epoxies, photoresists, polymers, silicon, silica, quartz, glass, controlled pore glass, carbon, and the like, and combinations thereof. Preferred insulative materials include thermoplastic polymers such as nylon, polypropylene, polyester, polycarbonate and the like. In some embodiments, the coating is a combination or mixture of a conductor and an insulator. For example, a metal structure may have a coating which is part conductor and part insulator and may or may not be in a desired pattern. Metal structures having a coating that is made of part insulator and part conductor in a pattern, such as a striped coating, may be used to generate flow patterns or mobilization fields of a desired pattern. In some embodiments, the metal structures have semiconductor coatings which are made conductive or nonconductive by an electrical contact functionally linked thereto. Suitable materials for the semiconductor coating include silicon, electroconducting conjugated polymers known in the art, and the like. The metal structures may be uncoated or partially uncoated, but since electroosmotic pumping performance will likely degrade over time as the metal becomes oxidized, coated metal structures are preferred.

Magnetic Metal Structures and Magnetic Field

As described herein, when the coated metal structures are placed in a magnetic field, the metal structures will magnetize and create local magnetic field gradients which manipulate, i.e. trap, isolate, confine, immobilize particles affected by magnetic force fields. When the magnetic field is on, the metal structures will become magnetized to their saturation point, thereby creating strong gradients in the magnetic field near the surface of the structures. The spatial pattern of the gradients depends on the orientation of the applied magnetic field. If the applied magnetic field is parallel to the axis of a cylindrical post, the gradient will be radially symmetric, and particles affected by magnetic fields, such as paramagnetic particles, will be uniformly captured against the surface of the post. If the magnetic field is perpendicular to the post axis, there will be a polar pattern of attachment of the particles to the surface of the magnetized metal structures. When the magnetic field is turned off, the metal structure will no longer be magnetized and particles affected by magnetic fields will not be manipulated. See Deng, et al. (2001) Appl. Phys. Lett. 80:461-463, which is herein incorporated by reference. After the magnet is removed, it is possible to further manipulate the particles, e.g. move the particles away from the metal structures, by subjecting the particles to another mobilization field.

Preferred particles that are affected by magnetic fields include polymer-coated microspheres having diameters of about 1 μm to about 10 μm and a superparamagnetic core of about 5% to about 40% iron oxide by weight, such as COMPEL magnetic beads (Bangs Laboratories, Fishers, Ind.) and others known in the art or commercially available. The magnetic metal structures may be made by methods known in the art. See e.g. See Deng, et al. (2001) Appl. Phys. Lett. 80:461-463 and Mirowski, et al. (2004) Appl. Phys. Lett. 84(10):1785-1788, which are herein incorporated by reference.

B1. Magnetic Metal Structures Having Conductor Coatings

Nickel structures having a diameter of about 10 to about 250 μm and height of about 10 μm to about 500 μm are formed on a substrate suitable for microfluidic devices using methods known in the art such as electroplating into an insulating stencil on a copper-coated insulating substrate. A preferred material for the insulating stencil is SU-8 photoresist (MicroChem Incorporated, Newton, Mass.), however, other insulating stencils known in the art may be used. Suitable materials for the insulating substrate include glass, quartz, polymer, alumina, oxide-coated silicon wafers, and the like may be used. After the metal coat is deposited, the stencil may be removed by methods known in the art including solvents, plasma etching, or ashing at high temperature to give freestanding nickel structures on a thin copper film. The copper film is then removed by methods known in the art, such as chemical etching or plasma etching, to leave electrically isolated nickel structures, i.e. structures between which an electrical current can not flow.

Insulating channel walls may be created by patterning SU-8 to the same height as the nickel structures on the first substrate and then bonding a second substrate thereon such as a transparent insulating glass or polymer lid. Alternatively, the channel walls may be formed on a second substrate by etching or molding an insulating glass, silicon, or polymer lid with the channel pattern at the same depth as the height of the nickel structures and then aligning and bonding the second substrate over the structures on the other first substrate.

In preferred embodiments, solid metal, preferably nickel, structures are electroplated on a polymer substrate and then coated with about 20 nm Ti, followed by about 200 nm of gold, or vice versa, using methods known in the art, including sputter-coat methods. Followed by aligning and bonding a polymer lid having fluid channels and drilled holes for fluid access. In alternative embodiments, the metal structures may be insulating structures having about a 200 nm metal, preferably nickel, coating made by methods known in the art and those described herein.

Solid metal, preferably nickel, structures may also be created by electrical discharge milling (EDM) to remove the material between posts on a bonded metal foil (thickness about 10 μm to about 500 μm); press-fitting metal, preferably nickel; wires into a soft insulating substrate or a hard insulating substrate having predrilled holes; conventional machining with a very small mill (diameter of about 0.005 inches or less); wet etching of a metal, preferably nickel, film through an etch-resistant mask, similar to the method of etching printed circuit boards with laminated copper foil; or other methods known in the art. These solid metal structures will typically be formed on the same substrate that will form the floor of the microfluidic channel, but it is also possible to form the metal structures on a different substrate and then transfer them to the microfluidic channel by preferential adhesion using methods known in the art. For example, nickel structures may be formed by wet-etching from a nickel foil that is loosely adhered to a Teflon-like substrate and then adhesives or thermal bonding may be applied at the tops of the structures to transfer the structures to a different substrate.

a. Magnetic Field then Electroosmotic Field

When the electric field is on, conductor coatings on the metal structures will cause the formation of microvortices in a quadrupolar pattern around each metal structure that may be used for mixing particles, i.e. ICEO mixing. For example, after particles, such as dense, large magnetic beads, have been manipulated, e.g. isolated in the magnetic field gradients described above, the magnetic field is turned off and the electric field is turned on to create microvortices which mix the magnetic beads in the surrounding fluid. Unlike nanoparticles or molecular species, the magnetic beads do not mix well by diffusion in short times because of their density and typical diameters of 1 μm or greater. In some embodiments, a specific binding agent is immobilized on the surfaces of the magnetic particles. As used herein, a “specific binding agent”, “receptor”, “capture agent” and “capture reagent” are used interchangeably to refer to an agent that specifically interacts with or binds to a ligand. As used herein, a “ligand” is used interchangeably with an “analyte” and refers to an atom, molecule, or ion that binds or interacts with a given receptor to form a complex. Depending on the chemistry, the receptor/ligand interaction may be reversible or irreversible. The extent of the binding depends on the affinity of the ligand to the receptor. In the presence of multiple ligands and receptors, the binding can be competitive (different ligands compete for the same receptor) or non-competitive (each ligand binds to a different receptor).

The specific binding agent can be immobilized on the substrate surface in a manner that provides for qualitative and/or quantitative determination of the analyte identity via interaction with the specific binding agent. See e.g. O'Brien, J., et al. (2000) Anal. Chem. 72:703, which is incorporated herein by reference. Receptors and ligands include biomolecules such as cellular receptors, peptides, polypeptides, proteins, antibodies, antigens, polynucleotides, polysaccharides, lipids, steroids, prostaglandins, prostacyclines, organic compounds, inorganic compounds, combinations thereof, and the like. As used herein, “affixed”, “attached”, “associated”, “conjugated”, “connected”, “coupled”, “immobilized”, “adsorbed”, and “linked” are used interchangeably and encompass direct as well as indirect connection, attachment, linkage, or conjugation, which may be reversible or irreversible, unless the context clearly dictates otherwise. As used herein, “specifically binds” refers to the specific interaction of one of at least two different molecules for the others compared to substantially less recognition of other molecules. Generally, the molecules have areas on their surfaces or in cavities giving rise to specific recognition between the two molecules. Exemplary of specific binding are antibody-antigen interactions, enzyme-substrate interactions, polynucleotide interactions, and so forth.

b. Electroosmotic Field then Magnetic Field

In a sample of fluid comprising nonmagnetic and magnetic particles, an electroosmotic field may be applied first to cause ICEO mixing of the different types of particles. This would be useful for immunoassays, where the magnetic particles are coated with an antibody and the nonmagnetic particles are natural particles, such as cells, antigen molecules, or viruses, that are specifically bound by antibodies immobilized on the magnetic particles. After mixing, the electric field is turned off and the magnetic field is turned on to capture and label the bound particles with a detectable label. As used herein, a “detectable label” refers to a molecule capable of detection using methods known in the art including radiography, fluorescence, chemiluminescence, enzymatic activity, absorbance, and the like. Detectable labels include radioisotopes, fluorophores, chromophores, enzymes, dyes, metal ions, ligands such as biotin, avidin, strepavidin and haptens, quantum dots, and the like.

c. Switching the Electroosmotic Field, and Between the Magnetic Field

In preferred embodiments, ICEO mixing is enhanced by abruptly switching the applied electric field between two directions differing by about 45 degrees which will split the vortices generated in the first step and send the entrained particles from the first vortex into two or more new vortices in the next step. In some embodiments, the preferred electric field is a square wave with an average value of about 0 V, with a peak-to-peak field strength of about 100 V/cm or greater, and a frequency between about 20 and about 500 Hz. In some embodiments, an alternating-current waveform is preferred to a DC signal since bubbles of electrolysis gases are prevented or inhibited from forming at the metal structures.

In alternative embodiments, mixing is enhanced by abruptly switching the applied electric field in combination with switching on and off the magnetic field. The movement of the magnetic particles that are attracted to the metal structures during magnetic capture will cause the other particles and fluid sample to be mixed. In preferred embodiments, the applied magnetic field is strong such that the magnetic particles move with greater force and at a faster rate thereby creating a more significant disruption in the previous fluid flows and microvortices.

d. Concurrent Electroosmotic and Magnetic Fields

An electroosmotic field and a magnetic field may be applied concurrently to produce a differential yet simultaneous effect on magnetic and nonmagnetic particles. For example, the relative magnitudes of the electric and magnetic fields is adjusted using methods known in the art in order to concentrate or capture magnetic particles at or near the surface of the metal structures and circulate the nonmagnetic particles past the metal structures by the ICEO-generated vortices resulting from the electric field. In alternative embodiments, a rotating magnetic field and an electroosmotic field may be concurrently applied in order to create two different types of mixing, ICEO mixing and magnetic bead (bar) stirring.

Some of the above magnetic field and ICEO combinations are exemplified the following exemplary immunoassay:

Immunoassay Using Magnetic Selection and ICEO Mixing

In some embodiments, the coated metal structures of the present invention may be used in immunoassays using the magnetic particles as a renewable, antibody-coated surface. For example, magnetic beads having an avidin-coated surface which is then functionalized with biotinylated antibodies which specifically bind an analyte, i.e. antigen, of interest. The beads are then placed in a carrier solution of 0.1 mM KCl, or another solution of similar conductivity. An external magnetic field is applied over an array of metal structures, e.g. magnetic posts, parallel to the axis of the structures. In preferred embodiments, the array has center-to-center structure spacing of 250 microns, and the diameter of the structures is about 200 microns. In preferred embodiments, the external magnetic field is created by an electromagnet, or a small permanent magnet such as a 5×5×5 mm NdFeB cube, which can generate a field of 2000 G near its surface. While the external magnetic field is applied, magnetic particles near the metal structures are captured from the carrier fluid and adhere to the surface of the metal structures. Next, with the magnetic field still on, the remaining volume of the fluid in the microchannel is replaced with an aliquot of a test sample which may contain the target antigen. The fluid flow is stopped, the magnetic field is removed, and an electric field is applied to induce ICEO mixing in order to circulate the magnetic beads throughout the test sample, thereby allowing any target antigens in the sample to bind the antibodies immobilized on the surface of the magnetic beads. Then another aliquot of the test sample or a second test sample may be assayed by turning off the electric field and applying the magnetic field in order to recapture the magnetic beads, remove the first test sample, and introduce the next sample. Repeating these steps using aliquots of a sample or multiple samples will increase assay sensitivity as antigen concentration bound by the antibodies increases and ICEO mixing allows the antibodies to circulate more uniformly in the fluid sample and come into contact with more antigens in the fluid.

After contact with the last desired sample, the beads are recaptured by the magnetic field and washed with a suitable solution such as water or a buffer. Then a labeling solution such as a fluorescent tagging solution is contacted with the bound antigens. The labels bind with the antigens to produce an observable signal. In preferred embodiments the labeling solution comprises fluorescent molecules or fluorescent beads conjugated to antibodies which specifically bind the antigens. ICEO mixing may be applied as described above to ensure binding of the antigens by the labels or to increase the observable signal by ensuring that the antigens are saturated with bound labels. Then the unbound labels are washed away by capturing the magnetic beads and rinsing with water or a buffer. Finally, the bound labels may be observed by methods known in the art including fluorescent microscopy or the like. In some embodiments, more than one antigen may be assayed by using two or more specific antibodies and two or more different labels. In some embodiments, electrophoresis is used to detect distinct peaks for each antigen, each antigen having a label bound thereto, or the cleaved labels themselves to indicate the presence of given antigens.

To decrease the complexity of the magnetic/electric field cycling process, a relatively weak magnet may be used to produce a trapping magnetic force sufficient to capture the magnetic beads at a slow fluid flow rate, but insufficient to hold the magnetic beads in a faster flow rate or the flow speed of the microvortices created by ICEO mixing. In these embodiments, cycling of the magnetic capturing and ICEO mixing may be conducted by turning the electric field on and off without having to modulate the applied magnetic field.

B2. Magnetic Metal Structures Having Insulator Coatings

Magnetic metal structures with insulating coatings may be used for combined iDEP trapping and magnetic trapping. Insulating coatings such as oxides also serve to isolate metal-sensitive reactions such as PCR or from other compounds that are reactive with metals. The magnetic metal structures having insulating coatings may be made using methods known in the art. Examples of compatible insulators include sputtered silicon oxides, insulating polymer coatings such as vapor-deposited parylene, and low-temperature plasma enhanced chemical vapor deposition (PECVD) nitrides. See e.g.

Harnett, et al. (2001) J. Vac. Sci. Technol. B 19 (6):2842-2845, and Elgaid, et al. (2004) Microelectronic Eng. 73-74:452-455, which are herein incorporated by reference.

It is noted that the magnetic metal structures may be insulating structures having metal magnetic coats. The insulating structures comprise an insulative material such as a polymer or glass. The insulative material of the insulating structures may be the same or different than the material of the insulator coatings.

a. Magnetic Field and iDEP Trapping Field or iDEP Streaming Field

i. iDEP Trapping Field

In some embodiments, a magnetic field is applied in order to build up a supply of trapped magnetic particles around the metal structures. Then the magnetic field is switched off and an electric field sufficiently strong to induce dielectrophoresis which overcomes electrokinesis, advection, diffusion, and electrostatic repulsion to concentrate and trap particles (an iDEP trapping field) is applied. Then the magnetic particles, as well as other particles present in the fluid, move to the local minima of the iDEP traps. See e.g. Cummings & Singh (2003) Anal. Chem. 75(18):4724-4731, and U.S. Published Patent Applications 20040026250 and 20040211669, which are herein incorporated by reference. The combination of the magnetic field with iDEP trapping allows both magnetic and nonmagnetic particles to be isolated in areas around the metal structures by two different mechanisms, e.g. two different characteristics, magnetism and induced dipole moments. In some embodiments, the iDEP trapping field is first applied and then the magnetic field is applied. In preferred embodiments, both the magnetic field and the iDEP trapping field are applied as trapping of particles, including both magnetic and nonmagnetic particles, will be enhanced as compared to only one applied field.

ii. iDEP Streaming Field

In some embodiments, a magnetic field is first applied in order to capture magnetic particles around the metal structures. Then the magnetic field is switched off and an electric field sufficiently strong to induce dielectrophoresis sufficient to overcome diffusion but slightly weaker than electrokinetic flow or advection (an iDEP streaming field) is applied to stream or flow any nonmagnetic particles in the fluid past the magnetic particles. See e.g. Cummings & Singh (2003) Anal. Chem. 75(18):4724-4731, and U.S. Published Patent Applications 20040026250 and 20040211669, which are herein incorporated by reference. The combination of the magnetic field with iDEP streaming allows the magnetic particles to be isolated in areas around the metal structures while nonmagnetic particles move past the trapped magnetic particles and metal structures. In some embodiments, the iDEP streaming field is first applied and then the magnetic field is applied. In preferred embodiments, the magnetic field and the iDEP streaming field are applied at the same time.

In situations where selective retention of the magnetic particles free of other particles are preferred, e.g. the magnetic particles have an analyte of interest bound thereon, when the magnetic particles are still captured near the metal structures in the presence of a magnetic field, the microchannel and captured magnetic particles are washed with a solution such as a buffer or water to rid the microchannel and magnetic beads from any contaminants. In some embodiments, iDEP streaming may occur during the wash step. In alternative embodiments, any iDEP field is removed during the wash step.

B3. Metal Structures having Semiconductor Coatings

Metal structures having semiconductor coatings may be made using methods known in the art. For example, the metal structures may be made by creating a nickel magnetic post array on a highly doped or metal-coated silicon wafer, then applying a thin (100 nm) conformal silicon dioxide coating, then applying a vapor-deposited organic semiconductor coating such as pentacene. See Someya, et al. (2002) Langmuir 18:5299, which is herein incorporated by reference. The semiconductor layer may be patterned by lithography to make the surface of each structure electrically isolated, for example, by removing the semiconductor layer everywhere except on the vertical surfaces of the posts. By applying an electric field between the conducting substrate and the surrounding fluid, the conductivity of the overlying semiconductor layer may be varied from insulating to conducting, thereby providing dielectrophoretic fields (iDEP trapping or streaming) and electroosmotic fields (ICEO mixing), respectively.

In some embodiments, the metal structures have a semiconductor coating which is made conductive or nonconductive based on an electrical contact inside the structure. These metal structures having semiconductor coatings may be used as insulating structures for iDEP trapping or iDEP streaming as described above and then switched to conducting structures for ICEO mixing and stirring. In these embodiments, the particles to be isolated need not be magnetic; however, any magnetic particles present may be further isolated by applying a magnetic field. In some embodiments, when the semiconductor coating is conducting, the coating is also magnetic.

a. Electroosmotic Field then iDEP Trapping Field or iDEP Streaming Field

Metal structures having semiconductor coatings according to the present invention may be used as conducting structures for ICEO mixing and stirring in an applied electric field where the semiconductor coatings are conductive. Where the metal structures are magnetic, ICEO mixing and stirring may be combined with magnetic trapping as described above.

The mechanism of iDEP is incompatible with a conductive surface, i.e. the surface of the structures must be insulative. Thus, the semiconductor coatings may be switched from conductive coatings to insulative coatings using methods known in the art in order to provide a dielectrophoretic field in the presence of an applied electric field. The dielectrophoretic field may be an iDEP trapping field or an iDEP streaming field. Where the metal structures are magnetic, the iDEP fields may be combined with magnetic trapping as described above.

In all the embodiments where two or more mobilization fields may be concurrently applied, the mobilization fields need not exist simultaneously, but may overlap. For example, a first mobilization field is applied, then a second mobilization field is applied and then either the first or the second or both mobilization fields may be removed such that there is a period wherein both mobilization fields were present.

In all the embodiments provided herein, fluid flow to or from the microchannel having the metal structures may be diverted or interrupted at desired periods in order to separate, mix, or manipulate different particles. For example, while certain particles are undergoing mixing or manipulation, preventing fluid flow to or from the microchannel may be desired. However, once the mixing has ended, fluid flow may desirably commence in order to wash captured particles from contaminants, introduce aliquots of more sample or different samples, and the like. Various methods and means for diverting or interrupting fluid flow in microchannels are known in the art and are contemplated herein. In some embodiments, a fluid sample may be passed through the microchannel having the metal structures and then held in a region for further use or reuse at a subsequent time after a different fluid sample is introduced into the microchannel.

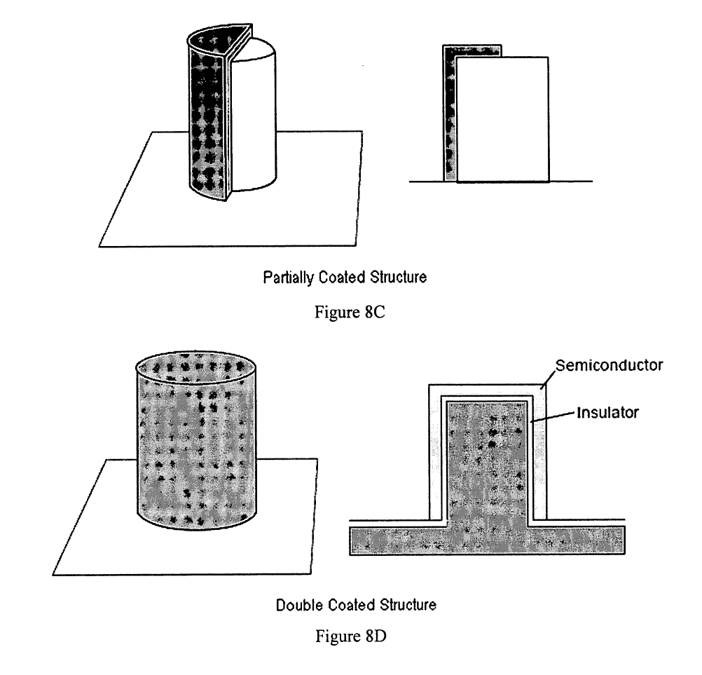

FIGS. 8A-8D show schematically show various types of metal structures and their cross-sections according to the present invention. The shapes and sizes of the metal structures are not limited to column like posts but include a variety of other shapes and sizes which may be readily selected by those skilled in the art for a desired application and result.

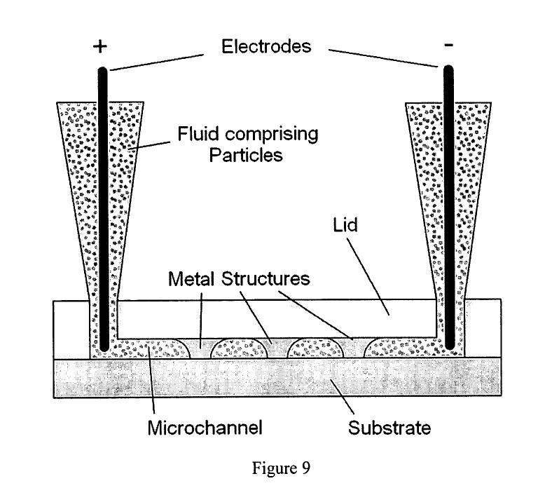

FIG. 9 schematically shows how the fields are applied to the metal structures. Various methods and means including a variety of electrodes and electrical contacts for applying a current to a microstructure or microchannel are known in the art and are contemplated herein. For example, electrical contact may be applied at wire electrodes inserted into the fluid channel, by direct electrical contact through insulated microfabricated metal traces that are connected to the conductive coating on individual microstructures, or by a similar microfabricated direct electrical contact to the conductive core of a solid metal post. Further, various geometries and configurations of microchannels and devices may, according to the present invention, be readily designed by one skilled in the art for desired versatility and performance.

Applications

Those skilled in the art will readily ascertain the various applications the devices and methods of the present invention may be employed. For example, the devices and methods of the present invention may be used to selectively combine and separate different types of particles to produce chemicals or make microstructured materials. Because of the low volume throughput of typical microfluidic devices, the target materials may be high value products such as pharmaceuticals, products such as toxic chemicals or explosives that are dangerous in large quantities, and experimental products such as new types of colloids to be used in electronics and displays. However, it is possible to scale up production by running microfluidic devices in parallel. See Nguyen (2005) Science 309:1021, which is herein incorporated by reference.

Some processes for making pharmaceutical products employ metallic elements, such as catalysts, which often have toxic effects and need to be removed. The devices and methods of the present invention may be used to mix the metallic catalysts and then remove the metallic elements to give a product free of metallic elements. See Hu, et al. (2005) J. Am. Chem. Soc. 127:12486-12487, which is herein incorporated by reference.

In some embodiments, the methods and devices of the present invention may be used to recover or reuse a desired agent such as a catalyst like an enzyme. The agent may be on a particle that may be captured using magnetic or iDEP fields induced by arrays of structures provided herein. In a catalyst-driven synthesis, ICEO-driven stirring may be used to mix the agent such that the agent interacts with more reactants in a shorter amount of time.

In addition to separating or handling one species of micro- or nanoparticle from a fluid of molecular-scale reactants, the methods and devices of the present invention may be used to manipulate a complex fluid having different types of particles (for instance, magnetic, dielectric, and particles having different surface charge, surface functionality, and diameters) with fine control over the presence or absence of each species of particle at a given step of the process.

Thus, the devices and methods of the present invention may be used to manufacture complex microparticle constructions. For example, one such construction which naturally appears in strong electric or magnetic fields is the particle chain that occurs by the alignment and mutual attraction of magnetizable particles in a magnetic field, or electric dipoles in a dielectrophoretic trap. If these particles are provided with a means of making a permanent connection, such as a photoactive epoxy coating, the chains may be linked into permanent structures and then expelled from the microfluidic device for further use.

Particle chains have been used for DNA separation, as self-assembled nanowires, and as micromagnetic stir bars. See e.g. Minc, et al. (2005) Electrophoresis 26:362-375, and Goubault, et al. (2005) Langmuir 21:3725-3729, which are herein incorporated by reference. Very complex structures may be built up by subsequent introduction and addition of particles having different properties. This is especially interesting if the new particles are added to the cluster in an asymmetric fashion rather than in a uniform coating, since asymmetric particles such as “Janus” spheres have been suggested for use in low-power electronic displays when having color-contrasting hemispheres and a permanent electric dipole moment. See Crowley, et al. (2002) J. Electrostatics 55:247-259, which is herein incorporated by reference.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

We claim:

1. A fluidic channel comprising at least one metal structure having a coat comprising a conductive material, an insulative material, a semi-conductive material, or a combination thereof.

2. The fluidic channel of claim 1, wherein the metal structure is an insulating structure having a metal coat.

3. The fluidic channel of claim 1, wherein the metal structure comprises solid metal.

4. The fluidic channel of claim 1, wherein the metal structure comprises a magnetic material, a paramagnetic material, or a ferromagnetic material.

5. The fluidic channel of claim 1, wherein the coat is about 1 nm to about 5000 nm thick.

6. The fluidic channel of claim 1, wherein the metal structure provides a magnetic field or an electroosmotic field under an applied electrical current.

7. The fluidic channel of claim 1, wherein the coat provides a mobilization field under an applied electrical current.

8. The fluidic channel of claim 7, wherein the mobilization field is an electroosmotic field, an iDEP field, or a combination thereof.

9. A method of manipulating or assaying a particle in a sample which comprises subjecting the sample to the fluidic channel of claim 1 and inducing at least one mobilization field.

10. The method of claim 9, wherein the mobilization field is an electroosmotic field.

11. The method of claim 9, wherein the metal structure comprises a magnetic material and the mobilization field is a magnetic field.

12. The method of claim 11, wherein the coat comprises a conductive material.

13. The method of claim 11, wherein the coat comprises an insulative material.

14. The method of claim 11, wherein the coat comprises a semi-conductive material.

15. The method of claim 12, which further comprises inducing an electroosmotic field.

16. The method of claim 13, which further comprises inducing an insulative dielectrophoresis (iDEP) field, which iDEP field may be an iDEP trapping field or an iDEP streaming field.

17. The method of claim 14, which further comprises inducing an electroosmotic field before or after inducing an iDEP field, which iDEP field may be an iDEP trapping field or an iDEP streaming field.

18. The method of claim 15, wherein the magnetic field and the electroosmotic field are induced concurrently or overlap.

19. The method of claim 16, wherein the magnetic field and the iDEP field are induced concurrently or overlap.

20. The method of claim 16, wherein either the electroosmotic field or the iDEP field is induced concurrently or overlap with the magnetic field.
